(19) United States
(12) Patent Application Publication
(10) Pub. No.: US 2004/0161735 A1
(43) Pub. Date: Aug. 19, 2004

(54) Cryopreservation of oocytes and embryos and methods for producing animals involving the same

(75) Inventors: Mark Brenton Nottle, South Australia (AU); Ranald Cameron, Queensland (AU); Luke Francis Sharkerley Beebe, Queensland (AU); Alan Weaver Blackshaw, Queensland (AU); Hiroshi Nagashima, Kanagawa (JP)

Correspondence Address: SUGHRUE MION, PLLC, 2100 PENNSYLVANIA AVENUE, N.W., SUITE 800, WASHINGTON, DC, 20037, US

(73) Assignees: THE UNIVERSITY OF QUEENSLAND; BRESAGEN LIMITED

(21) Appl. No.: 10/777,149

(22) Filed: Feb. 13, 2004

Related U.S. Application Data
(63) Continuation of application No. 09/856,689, filed on Sep. 26, 2001, now abandoned.
Application No. 09/856,689 is a 371 of international application No. PCT/AU99/01048, filed on Nov. 24, 1999.

(30) Foreign Application Priority Data
Nov. 24, 1998 (AU) PP 7299

(51) Int. Cl. A01N 1/02; A01K 67/027

(52) U.S. Cl. 435/2; 800/17

(57) ABSTRACT

A method for the cryopreservation of oocytes or embryos, which comprises centrifugation of oocytes or embryos to polarize cytoplasmic lipid outside the oocyte or embryonic cells, subjecting the oocytes or embryos to low temperature conditions in the presence of a cryoprotectant which results in freezing of the oocytes or embryos prior to lipid depolarization, followed by low temperature storage of the frozen lipid polarized oocytes or embryos are described, as are oocytes and embryos produced according to such methods, and methods for producing live animals.

The present invention relates to methods for the cryopreservation of oocytes and embryos, use of oocytes and embryos, and methods for producing live animals from such embryos. The production of live animals from frozen thawed embryos has been described for a number of species including cattle and sheep.

However the production of live animals such as piglets from frozen/thawed oocytes and embryos remains problematic. In relation to pigs for example, a sol number of piglets have been produced using conventional techniques and all have been from perihatching embryos, and not zona intact porcine embryos. The perihatching embryo stage is unsuitable for most uses because the embryos are not surrounded by an intact zona pellucida and are subject to bacterial and viral infection. This is an all important requirement for the import/export of genetic material. Such protocols require embryos to be surrounded by an intact zona pellucida because it protects against bacterial and viral infection as mentioned above, thus reducing the risk of disease transmission.

In other animals such as companion animals (for example dogs and cats), and domestic/livestock animals (for example horses, goats, llamas and alpacas), the production of live animals from frozen/thawed oocytes and embryos has been very problematic with a low success rate. This is largely because early stage embryos for most species contain significant levels of lipid.

The successful cryopreservation of animal oocytes and embryos, remains largely illusionary. In pigs in particular, cryopreservation techniques used for cryopreservation of embryos from other species are generally not successful.

In one prior proposal, animal embryos were subject to conditions which reduce the level of lipid in the embryo. In this proposal lipid was forced from the embryonic cells, resulting in a layer of lipid between the blastomeres and the zona pellucida. This lipid was moved by aspirating a lipid from the embryo using micro manipulation techniques. Removing lipids from embryos requires considerable technical skill, as well as much complexity.

The present invention seeks to overcome the problems associated with cryopreservation of oocytes and embryos, and seeks to provide simple, convenient and easily performed methods for the cryopreservation of oocytes and embryos, such as zona intact porcine embryos, and for producing live animals therefrom.

### SUMMARY OF INVENTION

In accordance with a first aspect of this invention there is provided a method for the cryopreservation of oocytes or embryos, which comprises centrifugation of oocytes or embryos to polarise cytoplasmic lipid outside the oocyte or embryonic cells, subjecting the oocytes or embryos to low temperature conditions in the presence of a cryoprotectant which results in freezing of the oocytes or embryos prior to lipid depolarisation, followed by low temperature storage of the frozen lipid polarised oocytes or embryos.

Preferably, the oocytes or embryos are vitrified by freezing in liquid nitrogen or other very cold fluid or gas which allows rapid temperature reduction.

In accordance with another aspect of this invention there is provided a method for producing animals from embryos which comprises thawing a cryopreserved lipid polarised embryo and thereafter transferring the embryo to a synchronized recipient female, and allowing the embryo to develop to term to give rise to live animals.

### DETAILED DESCRIPTION OF THE INVENTION

The present invention provides for the cryopreservation of animal embryos, for example zona intact porcine embryos, which hitherto have not been amenable to cryopreservation, and more parcularly the successful production of animals from the cryopreseved embryos. Cryopreserved cocytes can, on thawing, be fertilised, or genetically manipulated and fertilised, and then transferred into a pregnancy competent female to give live animals.

The inventors have surprisingly found that centrifugation of oocytes and embryos, such as zona intact porcine embryos. which polarises cytoplasmic lipid outside the oocyte and embryonic cells, followed by exposure to low temperature conditions, preferably vitrification, in the presence of cryoprotectant, enables successful cryopreservation of the polarised oocytes and embryos which maintains their viability such that on implantation into the uterus of a pregnancy competent female animals, progeny animals can develop. Accordingly, in a first aspect of this invention there is provided a method for the cryopreservation of animal oocytes embryonic cells comprising centrifugation of oocytes and embryos to polarise cytoplasmic lipid outside the oocyte or embryonic cells, subjecting the oocytes or embryos to low temperature conditions in the presence of a cryoprotectant which results in freezing of the embryo prior to lipid depolarisation, followed by low temperature storage of the frozen lipid polarised oocyte or embryo cells.

Embryos which may be subject to the methods of the present invention include zona intact embryos (blastomeres) from the oocyte stage, through to late blastocysts, including morulae to mid-blastocysts stage, and hatched (non-zona intact) blastocysts.

Oocytes and embryos may be from any animal, that is any mammal, including companion animals (for example dogs and cats), domestic/livestock animals (for example horses, cows, sheep, goats, pigs, llamas, and alpacas), laboratory animals (for example mice, rats, and monkeys), and humans. In a preferred aspect the invention relates to pigs, that is pig oocytes and embryos.

Oocytes and embryos may be recovered from donor animals by surgical or non-surgical methods. Non surgical methods can be used to recover oocytes and embryos from live cattle, but surgical methods are used for recovery from some other live animals, including pigs. For example, embryos may be surgically recovered from pigs within one to six days following mating. Alternatively, for livestock animals, oocytes and embryos may be flushed from reproductive tracts of slaughtered female animals. A second alternative is to obtain immature oocytes from the ovaries of slaughtered animals, and mature and fertilise them in vitro. The embryos obtained by any of these procedures may be briefly cultured in a medium standardly used for oocyte and/or embryo culture to an appropriate stage. Whilst in no way essential, it is generally desirable to briefly culture embryos prior to the methods of this invention.

The oocytes and embryos are cultured in a cryoprotectant-containing solution prior to vitrification. The oocyte and embryos need only be incubated in the cryoprotectant solution for a short period of time, for example from two minutes to one hour, more preferably from two minutes to 30 minutes, still more preferably from 3 minutes to 20 minutes.

Oocytes and embryos may be incubated in a cryoprotectant-containing solution either prior to, during or after centrifugation, or both.

The cryoprotectant-containing solution in which oocytes and embryos are incubated either prior to, during centrifugation, or after centrifugation, may contain any standard cryoprotectant established for use in the freezing of animal oocytes and embryos, including glycerol, ethylene glycol, dimethylsulfoxide, propylene glycol and polyvinyl pyrrolidine, sucrose, trehalose, Ficoll, acetamide, egg yolk and the like. The concentration of cryoprotectant is used in an amount sufficient to replace to at least some extent water within the oocyte or embryo, such that on rapid freezing ice crystal formation is prevented. By way of example, cryopreservatives may be present in an amount from 0.5M to 8M. One or more cryoprotectants may be used. The time in which oocytes and embryos may be incubated in a cryoprotectant solution following centrifugation is insufficient to allow lipid repolarisation into the tissues of the oocyte or embryo.

Oocytes and embryos are centrifuged for a time sufficient to polarise cytoplasmic lipid from the oocytes and embryonic cells to the outside of the cells, for example 1 to 15 minutes at 10,000 to 20,000 g. The time period of centrifugation will depend upon the centrifugal force applied during centrifugation. At a centrifugal force of about 13000 g polarisation takes place after about 8 minutes of centrifugation. It may be more convenient to centrifuge the oocytes and embryos in the presence of embryo culture medium, rather than in the presence of more viscous cryoprotective containing solutions.

Culture medium and cryoprotectant-containing solution for culture either before or after centrifugation may contain inhibitors of actin polymerisation such as Cytochalasin B which relaxes cytoskeletal elements.

Following centrifugation to polarise lipid, the lipid polarised oocytes and embryos are subject to low temperature conditions in the presence of a cryoprotectant which results in freezing of the oocyte or embryo prior to lipid depolarisation. By lipid depolarisation is meant the return of lipid to the cells which lipid was polarised outside the cells by centrifugation. It is to be noted that on polarisation cytoplasmic lipid may be attached to cells but displaced outside the cells.

Low temperature conditions may be provided by slow cooling, rapid freezing and vitrification. In these techniques the oocyte or embryo is frozen before the lipid returns to the cells. Vitrification may take place by placing the oocytes or embryos in a vessel, and plunging the vessel into an extremely cold environment, such as liquid nitrogen or other liquefied and/or gaseous extremely cold substance. Alternatively, a vessel containing oocytes or embryos may be rapidly frozen in an ultracold freezer, for example at temperatures below about −30° C. Any other apparatus or methods th enable rapid freezing may be used. In one example, oocytes or embryos may be loaded into a straw which is heat sealed, and then plunged into liquid nitrogen. In another example, embryos may be pulled by capillary action into a open pulled straw, which is then plunged into liquid nitrogen and subsequently stored (Vajta et al (1997) Cryoletters 18 191-5).

Oocytes and embryos may be stored in a conventional freezer facility, at temperatures, for example, from −10° C. to −70° C. or more.

Frozen lipid polarised oocytes or embryos may be thawed according to conventional oocyte and embryo thawing techniques, such as incubation of a frozen straw at a temperature of 35° C. to 39° C. in a suitable culture medium. Thawed embryos may be washed in culture medium, further cultured briefly, and then transferred to a sychronised recipient female, such as to the uterus of a pregnancy competent female animal. At the conclusion of pregnancy term, that is embryo development to term, the introduced embryos have developed to live animals.

The present invention provides a simple and straight forward procedure for cryopreservation of oocytes and embryos, particularly zona intact frozen embryos. On thawing and implantation of the embryos into the uterus of a pregnancy competent animal, animals may be produced in a manner which has not been achievable by the prior art.

In accordance with another aspect there is provided an animal when produced from an oocyte (subsequently fertilised) or embryo which has been cryopreserved in accordance with the invention hereinbefore described.

Oocytes or embryos may be subject to genetic manipulation prior to the process of this invention. In this regard one or more genes of interest may be inserted into an oocyte or embryo by established techniques, such as using pronuclear microinjection, homologous recombination using embryonic stem cell technologies and other established techniques for introducing genes into oocytes and embryos (Nottle et al (1997), Reprod Fertil Suppl 52, 237-244.

This invention allows banks or libraries of embryos or oocytes to be prepared. These banks or libraries may be provided in frozen form and presented in a convenient manner. Examples include a straw or tube, and a plurality of straws or tubes with multiple oocytes or embryos. The bank or library may contain oocytes or embryos from different animals and may find use in artificial insemination and breeding programs.

Certain embodiments of the present invention will now be exemplified with reference to the following non-limiting examples.

### EXAMPLE 1

Porcine embryos were collected and washed thoroughly in culture medium (mPB1-Quinn et al (1982) J. Reprod. Fert. 66:161-168) of 39° C. three times.

The embryos, in the early blastocyst stage were cultured for 40 minutes in the standard embryo culture medium NCSU-23 (Peters & Reed (1991), Theriogenology 35, 253) with 10% foetal bovine serum (FBS) containing 7.5μg/ml Cytochalasin B at 39° C. in a humidified environment of 5% CO₂ and air. After a five minute period of cooling from 39° C. to 25° C., the embryos were cultured for 5 minutes in 6% BSA in BECM-3h, and then washed in 25% VS3a for approximately three minutes (VS3a containing 6.5 m glycerol, 6% bovine serum albumin in BECM-3h (Dobrinsky et al (1996) Biol. Reprod. 55: 1069-1074). The embryos were centrifuged in a 1.5 Eppendorf tube (in the same media) at 13000 g for about 12 minutes, recovered back into 25% VS3a, and then left in that media for a further five minutes. The embryos were then washed for 30 seconds in 65% VS3a, followed by a wash in 1 ml VS3a before being loaded into a straw, heat sealing the straw, and plunging the embryos in the straw into liquid nitrogen.

In an alternative to storage in a heat-sealed straw, the embryos following a wash in the vitrification solution, are placed into a small drop of vitrification solution and drawn by capillary action into a narrowed hand pulled 0.25 ml freezing straw (unsealed pulled straw, UPS). The straw is then plunged directly into liquid nitrogen.

Results of one experiment are set forth in Table 1.

TABLE 1

| | | | | Number viable | |
|---|---|---|---|---|---|
| | Experi- | | | (%) with blastocoels | |
| Freezing | mental | Embryo | Embryo | after culture for | |
| Method | Replicates | Stage | Number | 24 h | 48 h |
| Standard | 2 | Mor | 4 | 0 | 0 |
| freezing method | 2 | MBI | 17 | 0 | 0 |
| (BEVS) | | | | | |
| | 2 | Peri | 13 | 2(15.4) | 2(15.4) |
| BEVS/Sp | 2 | Mor | 11 | 5(45.5) | 6(54.6) |
| | 3 | MBI | 43 | 27(62.8) | 26(60.5) |
| BEVS/UPS | 3 | Peri | 17 | 10(58.8) | 10(58.8) |
| BEVS/Sp/UPS | 2 | Mor | 6 | 3(50) | 5(83.3) |
| | 5 | MBI | 44 | 33(75) | 28(63.6) |

5 TABLE 1

Survival of Porcine Embryos frozen at various stages, thawed and then cultured for 48h. The morphological stages examined were Mor, morulae, MBI, early to mid blastocysts; Peri, peri-hatching blastocysts. The treatment used were BEVS, Beltsville embryo vitrification system (Dobrinsky et al (1997) Theriogenology 47:, 343); Sp, centrifuged, UPS, unsealed pulled straw.

### EXAMPLE 2

Method

Embryos were collected, washed thoroughly in mPB1 and then cultured for 35 minutes in NCSU-23+10% FBS with 7.5 μg/ml Cytochalasin B at 39° C. in an atmosphere of 5% CO₂ in air and 1001% humidity. Morulae to middle blastocyst stages were centrifuged at 13000 g for the last 10 minutes of this incubation in the culture medium containing the Cytochalasin B.

The embryos are then transferred to 2M ethylene glycol in mPB1 at 25° C. for five minutes before being washed thoroughly in 8M ethylene glycol and 7% PVP in PBS and then placed into a small droplet of the vitrification media and loaded into an unsealed pulled glass by capillary action. The straw is then plunged into liquid nitrogen and stored.

Thawing is by placing the end of the straw containing the embryos into 1.2 mls of 1M sucrose in PBS at 39° C. By blocking the open end with a finger, the fluid containing the embryos is forced out once, thawed, by the warming of the straw. Once collected, the embryos are placed into 1M ethylene glycol in mPB1 for two minutes followed by 0.5M ethylene glycol in mPB1 for a further 2 minutes, both at 25° C. Five minutes in MPB1 at 39° C. completes the rehydration procedure. The embryos can then be prepared for culture or transfer.

TABLE 2
Results

| | | | | Number Viable | |
|---|---|---|---|---|---|
| | | | | (%) with blastocoels | |
| Freezing | Embryo | | Embryo | after culture for | |
| Method | Stage | Replicates | Number | 24 hrs | 48 hrs |
| E/UPS | MBI | 1 | 5 | 0(0) | 0(0) |
| | Peri | 3 | 18 | 8(44.4) | 6(33.3) |
| E/Sp/UPS | MBI | 2 | 14 | 12(85.7) | 11(78.6) |

TABLE 2

Survival of freshly collected porcine embryos, vitrified with 8m ethylene glycol and 7% PVP. Embryos stages were early to middle blastocysts (MBI) and peri-hatching blastocysts (Peri). Freezing methods were: E/UPS, vitrified in the medium described above in an unsealed pulled straw, and E/Sp/UPS, vitrified in a similar manner but centrifuged at 13000 g during the last 10 minutes of the culture in NCSU-23+Cytochalasin B (7.5 μg/ml).

Transfer of Vitrified Embryos

The embryos once thawed using the appropriate technique are washed 3 times in mPB1 at 39° C. before being held in mPB1 until just prior to transfer. They are then washed in media (PBS+10% FBS+2% Penicillin/Strepomycin solution (CSL: Penicillin G 5000μ/ml.

Streptomycin sulphate 5000 μg/ml)) at 39° C. before loading into a Tomcat Catheter attached to a 1ml syringe followed by immediate transfer into one horn of the recipient animal.

TABLE 3

| Experi- | | | | | |
|---|---|---|---|---|---|
| ment | Day of | Embryonic | Embryo | Vitrification | Results |
| Number | Transfer | Stage | Number | Technique | thus far |
| 1 | 4 | Early | 37 | BEVS/Sp/UPS | Returned |
| | | blastocysts | | | |
| 2 | 4 | Early | 32 | BEVS/Sp/UPS | Returned |
| | | blastocysts | | | |
| 3 | 4 | Peri- | 37 | BEVS/UPS | Pregnant |
| | | hatching | | | 5 piglets |
| | | (16 hatched) | | | born alive |
| 4 | 4 | Peri- | 32 | BEVS/UPS | Pregnant |
| | | hatching | | | 3 piglets |
| | | | | | born alive |

TABLE 3

Results of the transfer of porcine embryos, vitrified and thawed, into pseudopregnant recipients. Techniques used were: BEVS, Beltsville Embryo Vitrification System: Sp, centrifugation as described in methods: UPS, vitrified and stored in an unsealed pulled straw. This recipient gave birth to 5 normal healthy piglets. A fourth recipient (Experiment 4) was also confirmed pregnant at 35 days gestation and produced 3 normal healthy piglets.

The two recipients from the Experiment 2 were continued as not pregnant 42 days after ovulation by progesterone assay. This was followed by a return to oestrus. The third recipient (Experiment 3) was confirmed as pregnant by progesterone assay 35 days after ovulation and by detection of a uterine artery pulse.

TABLE 4

| Recipient | Day of | Embryonic | Embryonic | |
|---|---|---|---|---|
| No. | Transfer | Stage | Number | Results |
| 1 | 4 | Early blastocyst | 36 | Pregnant |
| | | Zona intact | | 5 piglets born |
| | | | | alive |
| 2 | 4 | Early blastocyst | 36 | Delayed return |
| | | Zona intact | | |
| 3 | 4 | Early blastocyst | 38 | Delayed return |
| | | Zona intact | | |
| 4 | 4 | Early blastocyst | 37 | 21 day return |
| | | Zona intact | | |

TABLE4

Results of the transfer of zona intact early blastocysts. The embryos were centrifuged during the pre-incubation period with Cytochalasin B, vitrified with 8 methyleneglycol and 7% PVP while stored in unsealed pulled straws.

Throughout this specification, unless the context requires otherwise, the word “comprise”, or variations such as “comprises” or “comprising” or the term “includes” or variations thereof, will be understood to imply the inclusion of a stated element or integer or group of elements or integers but not the exclusion of any other element or integer or group of elements or integers. In this regard, in construing the claim scope, an embodiment where one or more features is added to any of claims is to be regarded as within the scope of the invention given that the essential features of the invention as claimed are included in such an embodiment.

1. A method for the cryopreservation of oocytes or embryos, which comprises centrifugation of oocytes or embryos to polarise cytoplasmic lipid outside the oocyte or embryonic cells, subjecting the oocytes or embryos to low temperature conditions in the presence of a cryoprotectant which results in freezing of the oocytes or embryos prior to lipid depolarisation, followed by low temperature storage of the frozen lipid polarised oocytes or embryos.

2. A method according to claim 1 wherein the embryos are zona intact embryos.

3. A method according to claim 1 wherein oocytes and embryos are obtained from companion animals (for example dogs and cats), and domestic/livestock animals (for example horses, cows, sheep, goats, llamas and alpacas).

4. A method according to claim 3 wherein the oocytes and embryos are porcine oocytes and embryos.

5. A method according to claim 1 wherein the embryos are vitrified in the presence of a solution containing one or more cryoprotectant agents.

6. A method according to claim 2 wherein the cryoprotectants are selected from dimethylsulfoxide, propylene glycol, ethylene glycol, glycerol, PVP, sucrose, trehalose, Ficoll, acetamide and egg yolk.

7. A method for producing live animals from embryos which comprises thawing a cryopreserved zona intact embryo produced according to claim 1 and thereafter introducing the embryo into the uterus or fallopian tubes of a pregnancy competent female pig which at the conclusion of pregnancy term gives rise to live piglets.

8. An animal produced form a cryopreserved oocyte or embryo produced according to claim 1.

9. An animal produced according to the method of claim 7.

10. An animal according to claim 7 which is a pig.

11. A cryopreserved oocyte or embryo when produced according to claim 1.

12. A method according to claim 3 wherein embryos are vitrified by freezing in liquid nitrogen.

13. A method according to claim 10 wherein oocytes or embryos are frozen in a freezing vessel including cytologic vials or freezing straws including open pulled straws in which the oocytes or embryos are located by capillary action.

14. A method according to claim 2 wherein the embryos are in the morulae to mid-blastocyst stage.
